(19) United States
(12) Patent Application Publication
(10) Pub. No.: US 2026/0090731 A1
(43) Pub. Date: Apr. 2, 2026

(54) RING-TYPE ELECTRODERMAL ACTIVITY MEASUREMENT DEVICE

(71) Applicant: PIXART IMAGING INC., Hsin-Chu County (TW)

(72) Inventors: Chih-Hao WANG, Hsin-Chu County (TW); Shih-Jen LU, Hsin-Chu County (TW); Yang-Ming CHOU, Hsin-Chu County (TW); Chien-Yi KAO, Hsin-Chu County (TW); Hung-Chih WANG, Hsin-Chu County (TW); Hsin-Yi LIN, Hsin-Chu County (TW)

(21) Appl. No.: 18/904,088

(22) Filed: Oct. 2, 2024

(51) Int. Cl. A61B 5/0531; A61B 5/00

(52) U.S. Cl. CPC A61B 5/0531; A61B 5/0002; A61B 5/6826

(57) ABSTRACT

There is provided a ring-type electrodermal activity measurement device including a first electrode, a second electrode and a sensor chip. The first electrode is for contacting a first skin of a first finger, and for receiving a first voltage. The second electrode is for contacting a second skin of a second finger, different from the first finger, and for receiving a second voltage. The sensor chip is coupled to the first electrode and the second electrode, and is to measure a voltage variation of a skin area between the first electrode and the second electrode to detect the electrodermal activity.

### FIELD OF THE DISCLOSURE

This disclosure generally relates to a physiological measurement device and, more particularly, to an electrodermal activity measurement device that arranges two electrodes on a ring-type structure for contacting skins of two different fingers, respectively.

### BACKGROUND OF THE DISCLOSURE

The present electrodermal activity (EDA) sensor is arranged on a watch. The two electrodes for measuring signals are both arranged on a front surface or an inner surface of the watch.

When the two electrodes are arranged on the front surface of a watch, a user uses his/her palm of the hand not wearing the watch to contact the two electrodes to perform the measurement. This arrangement is mainly for short-term measurement, but is not suitable for the long-term measurement.

In the case that the two electrodes are arranged on the inner surface of a watch, the two electrodes can continuously contact a surface of wrist. However, because the sweat gland density on the wrist skin is low, the obtained signal quality for EDA measurement is poor.

The information disclosed in this BACKGROUND is merely intended to increase understanding of the general background of the invention and should not be taken as an admission or in any way implied that the relevant information constitutes prior art that is already known to a person of ordinary skill in the art.

### SUMMARY

Accordingly, the present disclosure provides a ring-type physiological measurement device for wearing on a user's finger to measure the electrodermal activity. Because user's fingers generally have higher sweat gland density, high signal quality for EDA measurement is obtainable.

The present disclosure provides a ring-type physiological measurement device with two electrodes for contacting skins of two different fingers, respectively. The electrodermal activity is measured by detecting a voltage change of a skin area between the two electrodes.

The present disclosure provides an electrodermal activity measurement device to be worn on a finger of a user, and including a ring body, a first electrode, a second electrode and an isolation zone. The ring body includes an outer surface and an inner surface. The first electrode is arranged at a first zone on the outer surface, and configured to contact a first finger of the user. The second electrode is arranged at a second zone on the outer surface, and configured to contact a second finger of the user. The isolation zone is arranged on the outer surface, and configured to electrically isolate the first electrode and the second electrode.

The present disclosure further provides an electrodermal activity measurement device to be worn on a finger of a user, and including a ring body, a first electrode and a second electrode. The ring body includes an outer surface and an inner surface. The first electrode is arranged at a first zone on the outer surface, and configured to contact a first finger of the user. The second electrode is arranged at a second zone on the inner surface, and configured to contact a second finger of the user.

The present disclosure further provides an electrodermal activity measurement device including a first ring body, a second ring body and a sensor chip. The first ring body is to be worn on a first finger of a user, and includes a first electrode arranged on one of an inner surface and an outer surface of the first ring body and configured to contact the first finger or an adjacent finger of the first finger. The second ring body is to be worn on a second finger of the user, and includes a second electrode arranged on one of an inner surface and an outer surface of the second ring body and configured to contact the second finger or an adjacent finger of the second finger. The sensor chip is arranged on the first ring body or the second ring body, and electrically coupled to the first electrode and the second electrode.

### BRIEF DESCRIPTION OF DRAWINGS

Other objects, advantages, and novel features of the present disclosure will become more apparent from the following detailed description when taken in conjunction with the accompanying drawings.

FIG. 1 is a schematic diagram of an electrodermal activity measurement device according to one embodiment of the present disclosure.

FIG. 2A is a solid diagram of an electrodermal activity measurement device according to a first embodiment of the present disclosure.

FIG. 2B is a wearing schematic diagram of an electrodermal activity measurement device according to a first embodiment of the present disclosure.

FIG. 2C is a solid diagram of an electrodermal activity measurement device according to an alternative first embodiment of the present disclosure.

FIG. 2D is a solid diagram of an electrodermal activity measurement device according to an alternative first embodiment of the present disclosure.

FIG. 3A is a solid diagram of an electrodermal activity measurement device according to a second embodiment of the present disclosure.

FIG. 3B is a wearing schematic diagram of an electrodermal activity measurement device according to a second embodiment of the present disclosure.

FIG. 3C is a solid diagram of an electrodermal activity measurement device according to an alternative second embodiment of the present disclosure.

FIG. 4A is a solid diagram of an electrodermal activity measurement device according to a third embodiment of the present disclosure.

FIG. 4B is a wearing schematic diagram of an electrodermal activity measurement device according to a third embodiment of the present disclosure.

FIG. 4C is a solid diagram of an electrodermal activity measurement device according to an alternative third embodiment of the present disclosure.

FIG. 4D is a solid diagram of an electrodermal activity measurement device according to a further alternative third embodiment of the present disclosure.

### DETAILED DESCRIPTION OF THE DISCLOSURE

It should be noted that, wherever possible, the same reference numbers will be used throughout the drawings to refer to the same or like parts.

One objective of the present disclosure is to provide a ring-type measurement device that is used for the long-term measurement of the electrodermal activity (EDA) continuously. The ring-type measurement device includes two electrodes respectively for contacting two different fingers, which are fingers of the same or different hands of a user according to the using habits of the user.

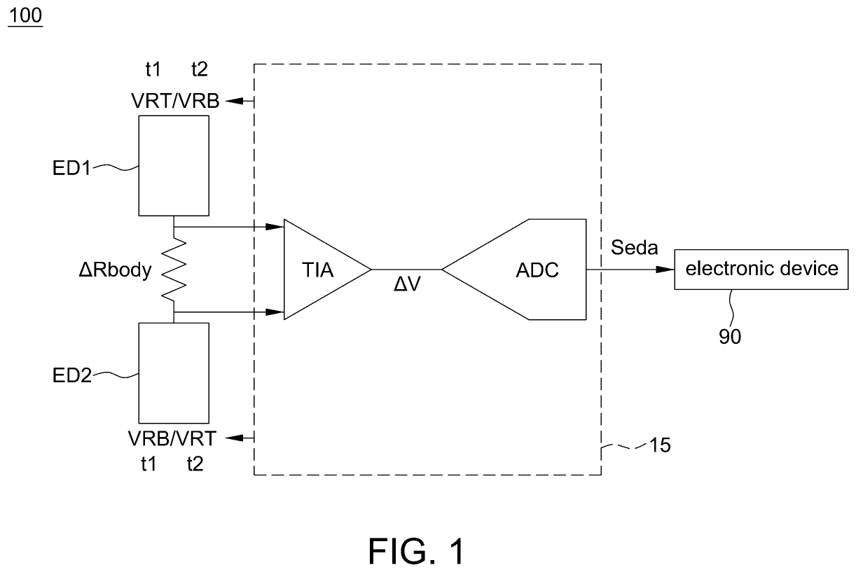

Please refer to FIG. 1, it is a schematic diagram of an electrodermal activity measurement device 100 (abbreviated as measurement device 100 herein) according to one embodiment of the present disclosure. The measurement device 100 includes a first electrode ED1, a second electrode ED2 and a sensor chip 15. The first electrode ED1 and the second electrode ED2 are used to contact skins of different fingers (may be the same or different hands) of a user. The sensor chip 15 is, for example, an application specific integrated circuit (ASIC), a digital signal processor (DSP) or a field programmable fata array (FPGA), and used to provide different voltages to the first electrode ED1 and the second electrode ED2, and to measure a resistor change ΔRbody between the first electrode ED1 and the second electrode ED2. The sensor chip 15 represents the sensor chips in the following embodiments.

In one aspect, to prevent the first electrode ED1 and the second electrode ED2 from being polarized, the sensor chip 15 provides a first voltage VRT to the first electrode ED1 and provides a second voltage VRB, different from the first electrode VRT, to the second electrode ED2 in a first time interval t1; and provides the second voltage VRB to the first electrode ED1 and provides the first voltage VRT to the second electrode ED2 in a second time interval t2, wherein a length of the first time interval t1 is identical to or different from that of the second time interval t2. In other words, the sensor chip 15 alternatively provides a high voltage (e.g., VRT) and a low voltage (e.g., VRB) to the first electrode ED1 and the second electrode ED2, respectively. Values of the first voltage VRT and the second voltage VRB are previously determined according to voltages generally utilized to measure the EDA.

It is known that a skin resistor Rbody between the first electrode ED1 and the second electrode ED2 can change due to sweat amount on a skin surface between the first electrode ED1 and the second electrode ED2 to cause a resistor change ΔRbody, which can reflect the EDA. In one aspect, the sensor chip 15 includes, for example, a trans-impedance amplifier (TIA) for converting and amplifying current signals to voltage signals such that the resistor change ΔRbody correspondingly generates a voltage change ΔV. The sensor chip 15 further includes an analog-to-digital converter (ADC) to generate digitized voltage signals. The sensor chip 15 is able to detect the EDA according to these digitized voltage signals. The method of measuring the EDA according to the voltage change ΔV is known to the art, and thus details thereof are not described herein.

In the present disclosure, the sensor chip 15 preferably further includes a wireless communication chip (e.g., an RF chip, a BT chip, but not limited to) for transmitting measurement data Seda to an external electronic device 90 in a wireless manner. The electronic device 90 is, for example, a smart cellphone or a smart watch, but not limited thereto. The measurement data Seda may be a voltage change, digitized voltage value or measurement result of the EDA based on functions of the sensor chip 15. The electronic device 90 performs predetermined controls according to the received measurement data Seda corresponding to different applications.

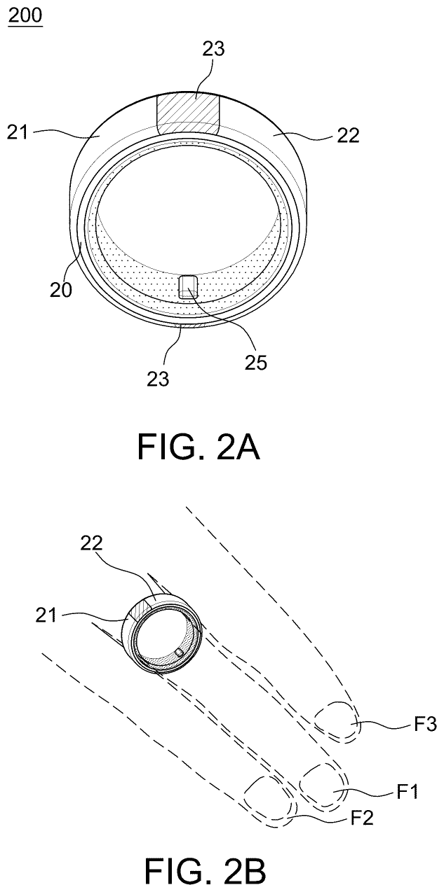

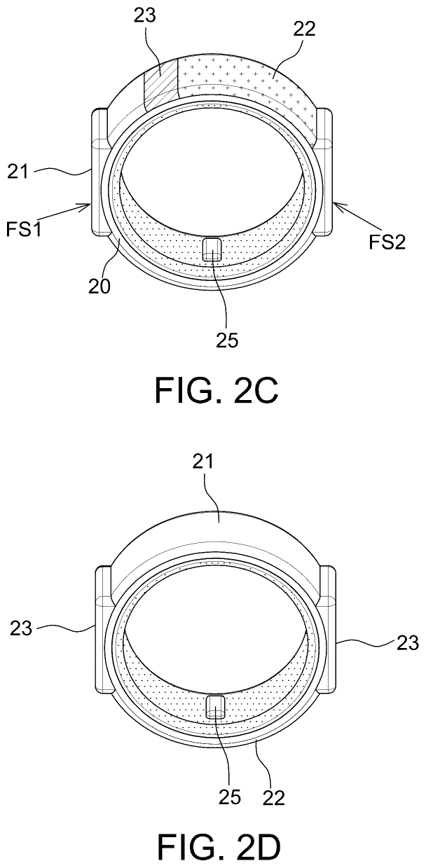

Please refer to FIGS. 2A and 2B, FIG. 2A is a solid diagram of an electrodermal activity (EDA) measurement device 200 according to a first embodiment of the present disclosure; and FIG. 2B is a wearing schematic diagram of an EDA measurement device 200 according to a first embodiment of the present disclosure. The EDA measurement device 200 (abbreviated as measurement device 200) is used to be worn on a user's finger and has a ring-type structure, e.g., including a ring, finger cots or brass knuckles, but not limited thereto.

The measurement device 200 includes a ring body 20, a first electrode 21, a second electrode 22, an isolation zone 23 and a sensor chip 25. The sensor chip 25 is electrically coupled to the first electrode 21 and the second electrode 22 to provide the first voltage VRT and the second voltage VRB, and to measure a voltage change ΔV.

The ring body 20 includes an outer surface and an inner surface (i.e. the surface attaching to a wearing finger). In FIG. 2A, although the sensor chip 25 is shown to be arranged on the inner surface, it is only intended to illustrate but not to limit the present disclosure. In one aspect, the sensor chip 25 is embedded inside the ring body 20 and not exposed outside a surface of the ring body 20.

The first electrode 21 is arranged at a first zone on the outer surface, and used to contact a first finger of the user, e.g., shown as a finger F2. The second electrode 22 is arranged at a second zone on the outer surface, and used to contact a second finger of the user, e.g., shown as a finger F3. The isolation zone 23 is arranged on the outer surface, and used to electrically isolate the first electrode 21 and the second electrode 22. A size of the isolation zone 23 is not limited to those shown in FIGS. 2A and 2B as long as the first electrode 21 and the second electrode 22 are electrically isolated thereby. In one aspect, the isolation zone 23 is arranged to have different features from the first electrode 21 and the second electrode 22 (e.g., having different colors or with a predetermined mark thereon) such that the user takes it as a reference to wear the measurement device 200 on a finger at a predetermined angle/direction. For example, in the aspect of FIG. 2A-2B, the isolation zone 23 is directed to upward as a way of correctly wearing the measurement device 200.

It should be mentioned that although FIG. 2A shows that a surface of the isolation zone 23 has the same height and curvature as those of surfaces of the electrodes 21 and 22 (forming a smooth connection interface therebetween), the present disclosure is not limited thereto. In another aspect, the surface of the isolation zone 23 is arranged to be higher than or lower than the surfaces of the electrodes 21 and 22 (e.g., forming said different feature).

It should be mentioned that although FIGS. 2A and 2B show that surfaces of the first electrode 21 and the second electrode 22 are curved surfaces, the present disclosure is not limited thereto. In another aspect, a surface of the first electrode 21 and the second electrode 22 is respectively a flat surface, e.g., FS1 and FS2 shown in FIG. 2C, which is suitable to be in contact with an adjacent finger of a wearing finger (e.g., shown as F1). By arranging surfaces of the first electrode 21 and the second electrode 22 as flat surfaces, the user is easier to wear the measurement device 200 at a predetermined angle/direction. In another aspect, the surface of the first electrode 21 and the second electrode 22 is respectively a concave surface, or a convex surface with a different curvature from that of the outer surface of ring body.

In one aspect, the first zone and the second zone are arranged in the way that when the ring body 20 is worn on a predetermined finger F1 of the user at a predetermined angle/direction, the first electrode 21 and the second electrode 22 are respectively in contact with two adjacent fingers F2 and F3 at two sides of the predetermined finger F1 as shown in FIG. 2B.

In another aspect, the first zone and the second zone are arranged in the way that when the ring body 20 is worn on a predetermined finger F1 of the user at a predetermined angle/direction, one of the first electrode 21 and the second electrode 22 (e.g., the first electrode 21) is in contact with one of two adjacent fingers (e.g., F2) at two sides of the predetermined finger F1, but the other one of the first electrode 21 and the second electrode 22 (e.g., the first electrode 22) is not in contact with the other one of the two adjacent fingers (e.g., F3) at two sides of the predetermined finger F1. In FIG. 2C, when the ring body 20 is worn on a predetermined finger F1 of the user at a predetermined angle/direction, the second electrode 22 is located at a top of the ring body 20 (not extending to the flat surface FS2) and thus is not in contact with the finger F3. When the user is going to measure the EDA, the user uses any finger of the non-wearing hand to touch the second electrode 22 such that the first electrode 21 and the second electrode 22 are respectively in contact with fingers of different hands.

In another aspect, the outer surface of the ring body 20 has a single flat surface, e.g., FS1 is a flat surface and FS2 is changed to a curved surface as shown in FIG. 2A. In this aspect, the second electrode 22 is arranged as FIG. 2A or FIG. 2C.

In a further aspect, the first zone and the second zone are arranged in the way that when the ring body 20 is worn on a predetermined finger F1 of the user at a predetermined angle/direction, none of the first electrode 21 and the second electrode 22 is in contact with adjacent fingers at two sides of the predetermined finger F1. In this aspect, the user uses two fingers (not limited) of the other hand (i.e. non-wearing hand) to respectively touch the first electrode 21 and the second electrode 22 to perform the measurement.

For example, FIG. 2D shows that the first electrode 21 and the second electrode 22 are respectively located at a top surface (or bottom surface) and a bottom surface (or top surface) when the ring body 22 is worn on a predetermined finger F1 at an angle/direction shown in FIG. 2D. In this aspect, two isolation zones 23 are arranged at two sides of the ring body 22 in contact with two fingers at two sides of the predetermined finger F1. The sizes of the first electrode 21 and the second electrode 22 are not particularly limited as long as the first electrode 21 and the second electrode 22 are not in contact with the two fingers at two sides of the predetermined finger F1 when the ring body 22 is worn on a predetermined finger F1 at an angle/direction shown in FIG. 2D.

In another aspect, both the first electrode 21 and the second electrode 22 are located only at a top surface or a bottom surface of the ring body 22, and separated by an isolation zones 23 similar to in FIG. 2A. However, the first electrode 21 and the second electrode 22 do not extend to the two sides of the ring body 22 in contact with two fingers at two sides of the predetermined finger F1.

More specifically, in the first embodiment, the first electrode 21 and the second electrode 22 are both arranged at the outer surface of the ring body 20, and are isolated by the isolation zone 23. The material of the isolation zone 23 is not particular limited.

It should be mentioned that although FIG. 2C shows that a surface of the isolation zone 23 has the same height and same curvature as those of a surface of the second electrode 22 (e.g., forming a smooth connection interface therebetween), the present disclosure is not limited thereto. In another aspect, the surface of the isolation zone 23 is arranged to be higher than or lower than the surface of the second electrode 22 (e.g., forming said different feature).

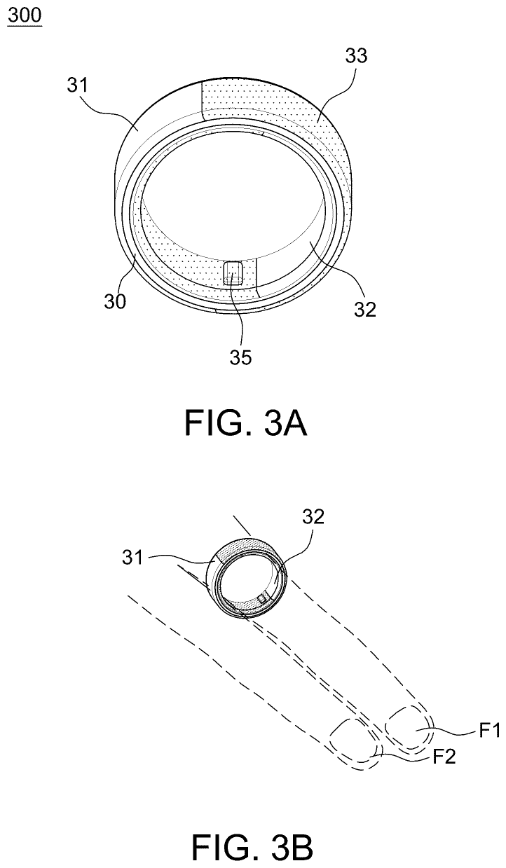

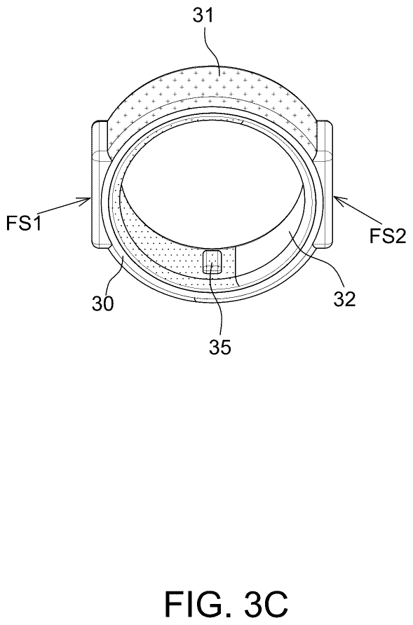

Please refer to FIGS. 3A and 3B, FIG. 3A is a solid diagram of an electrodermal activity (EDA) measurement device 300 according to a second embodiment of the present disclosure; and FIG. 3B is a wearing schematic diagram of an EDA measurement device 300 according to a second embodiment of the present disclosure. The EDA measurement device 300 (abbreviated as measurement device 300) is used to be worn on a user's finger and has a ring-type structure, e.g., including a ring, finger cots or brass knuckles, but not limited thereto.

The measurement device 300 includes a ring body 30, a first electrode 31, a second electrode 32, an isolation zone 33 and a sensor chip 35. The sensor chip 35 is electrically coupled to the first electrode 31 and the second electrode 32 to provide the first voltage VRT and the second voltage VRB, and to measure a voltage change ΔV.

The ring body 30 includes an outer surface and an inner surface. In FIG. 3A, although the sensor chip 35 is shown to be arranged on the inner surface, it is only intended to illustrate but not to limit the present disclosure. In one aspect, the sensor chip 35 is embedded inside the ring body 30 and not exposed outside a surface of the ring body 30.

The first electrode 31 is arranged at a first zone on the outer surface, and used to contact a first finger of the user, e.g., shown as a finger F2. The second electrode 32 is arranged at a second zone on the inner surface, and used to contact a second finger of the user, e.g., shown as a finger F1. The second zone convers a part of or the whole of the inner surface without particular limitations.

The isolation zone 33 is arranged at the rest zone outside the first zone on the outer surface to prevent an adjacent finger (not shown) at the other side of the second finger F1 from contacting the first electrode 31. Similarly, the isolation zone 23 may be arranged to have different features from the first electrode 31 (e.g., having different colors or with a predetermined mark thereon) such that the user takes it as a reference to wear the measurement device 300 on a finger at a predetermined angle/direction. Similarly, the material of the isolation zone 33 is not particular limited.

In one aspect, the first zone is arranged in the way that when the ring body 30 is worn on the second finger F1 of the user at a predetermined angle/direction, the first electrode 31 is in contact with one of two adjacent fingers (e.g., F2) at two sides of the second finger F1 but is not in contact with the other one of the two adjacent fingers at two sides of the second finger F1. A surface of the first electrode 31 is a curved surface (e.g., FIG. 3A) or a flat surface (e.g., FIG. 3C).

In another aspect, the first zone is arranged in the way that when the ring body 30 is worn on the second finger F1 of the user at a predetermined angle/direction, the first electrode 31 is not in contact with any one of two adjacent fingers at two sides of the second finger F1. For example FIG. 3C shows that when the ring body 30 is worn on the second finger F1 of the user at a predetermined angle/direction, the first electrode 31 is located at a top of the ring body 30 (not extending to FS1 and FS2) such that the first electrode 31 is not in contact with fingers at two adjacent sides. When a user performs the measurement, the user may use any finger of the non-wearing hand or another finger of the wearing hand (if possible) to touch the first electrode 31 such that the first electrode 31 and the second electrode 32 are respectively in contact with fingers of different hands. Or, the user may rotate the ring body 30 to cause the first electrode 31 to contact one of two adjacent fingers at two sides of the wearing finger F1 according to the using habits of the user.

Similarly, the outer surface of the ring body 30 may have a single flat surface, e.g., FS1 (the side arranging the first electrode 31) being a flat surface and FS2 (the side not arranging the first electrode 31) being changed to a curved surface as shown in FIG. 3A, That is, in the second embodiment, when the ring body 30 is worn on the second finger F1 of the user at a predetermined angle/direction, two opposite sides of the outer surface of the ring body 30 corresponding to two adjacent fingers at two sides of the second finger F1 are curved surfaces or flat surfaces.

Positions of the first electrode 31 and the isolation zone 33 shown in FIG. 3A may be exchanged to allow the first electrode 31 to contact a finger (not shown) at the other side of the wearing finger F1.

In an alternative embodiment, the first embodiment and the second embodiment are combinable. For example, an electrodermal activity measurement device of the present disclosure includes three electrodes, e.g., 21, 22 and 32, referring to FIGS. 2A, 2C, 3A and 3C. Two among the three electrodes are at the outer surface and one among the three electrodes is at the inner surface of a ring body of the electrodermal activity measurement device. Two among the three electrodes are driven to perform the measurement. For example, in a first time interval the electrodes 21 and 22 are driven to perform the measurement, and in a second time interval the electrodes 21 (or 22) and 32 are driven to perform the measurement. In one aspect, the first time interval and the second interval are arranged alternatively, but not limited thereto, the first time interval and the second interval are switched according to a predetermined arrangement.

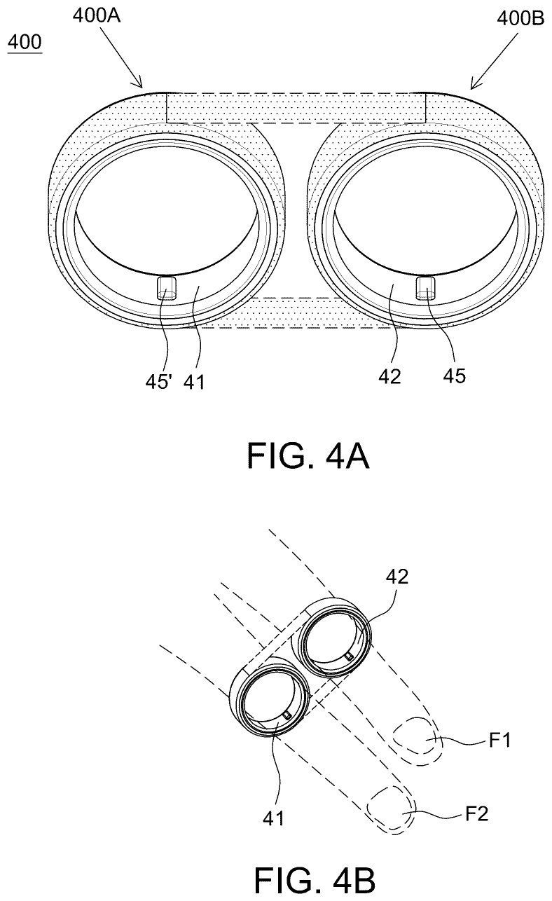

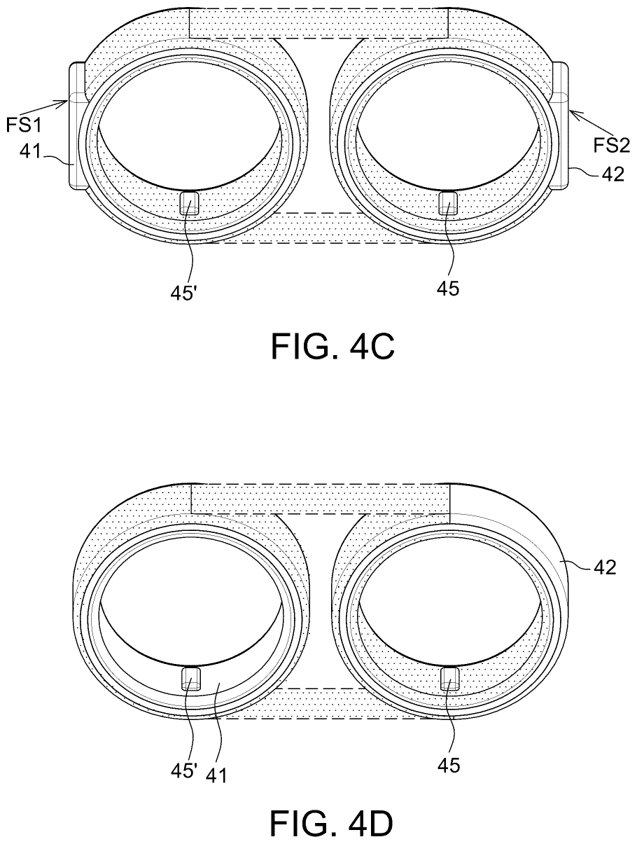

Please refer to FIGS. 4A and 4B, FIG. 4A is a solid diagram of an electrodermal activity (EDA) measurement device 400 according to a third embodiment of the present disclosure; and FIG. 4B is a wearing schematic diagram of an EDA measurement device 400 according to a third embodiment of the present disclosure. The EDA measurement device 400 (abbreviated as measurement device 400) includes a first ring body 400A, a second ring body 400B and a sensor chip 45.

The first ring body 400A is used to be worn on a first finger (e.g., shown as F2) of a user, and includes a first electrode 41 arranged on one of an inner surface (e.g., FIG. 4A) and an outer surface (e.g., FIG. 4C) of the first ring body 400A, and is used to contact a first finger F2 or an adjacent finger of the first finger F2, not the finger F1.

The second ring body 400B is used to be worn on a second finger (e.g., shown as F1) of the user, and includes a second electrode 42 arranged on one of an inner surface (e.g., FIG. 4A) and an outer surface (e.g., FIG. 4C) of the second ring body 400B, and is used to contact a second finger F1 or an adjacent finger of the second finger F1, not the finger F2.

When the first electrode 41 is arranged at the outer surface of the first ring body 400A, the first electrode 41 is arranged at an opposite side of the second ring body 400B, e.g., FS1 side in FIG. 4C. When the second electrode 42 is arranged at the outer surface of the second ring body 400B, the second electrode 42 is arranged at an opposite side of the first ring body 400A, e.g., FS2 side in FIG. 4C. Similarly, in this aspect, a surface of the first electrode 41 is a flat surface or a curved surface; and a surface of the second electrode 42 is a flat surface or a curved surface.

When the first electrode 41 is arranged at the inner surface of the first ring body 400A, the first electrode 41 covers a part of or the whole of the inner surface of the first ring body 400A as long as it has enough area in contact with the wearing finger. When the second electrode 42 is arranged at the inner surface of the second ring body 400B, the second electrode 42 covers a part of or the whole of the inner surface of the second ring body 400B as long as it has enough area in contact with the wearing finger.

FIG. 4D shows that one of the first electrode 41 and the second electrode 42 is arranged on the inner surface of the corresponding ring body and the other one is arranged at the outer surface of the corresponding ring body. In FIG. 4B, the wearing fingers of the first ring body 400A and the second ring body 400B may be exchanged.

The sensor chip 45 is electrically coupled to the first electrode 41 and the second electrode 42 to provide the first voltage VRT and the second voltage VRB, and to measure a voltage change ΔV of the detected skin surface.

In one aspect, the measurement device 400 further includes another chip 45′ which is used to operate in conjunction with the sensor chip 45, e.g., coupled wirelessly to each other. For example, when the sensor chip 45 provides the first voltage VRT to the second electrode 42, the chip 45′ provides the second voltage VRB to the first electrode 41, and vice versa. One of the sensor chip 45 and the chip 45′ is used to detect the voltage change ΔV of the detected skin surface.

Similarly, in another aspect, the sensor chip 45 and the chip 45′ are embedded inside the corresponding ring body and not exposed on the surface.

Preferably, the first ring body 400A and the second ring body 400B are connected/bonded together using the elastic component(s), whose material is not particularly limited.

It should be mentioned that although the present disclosure is described in the way that the electrodes are to contact a finger skin, the present disclosure is not limited thereto. In other aspects, the electrode arranged at the outer surface of the ring body is used to contact a palm skin depending on the using habits of a user, and preferably the two electrodes are not used to contact the skin of the same finger.

It should be mentioned that although the present disclosure is described in the way that the sensor chip is to measure a voltage change as a way to detect the EDA as an example, the present disclosure is not limited thereto. In other aspects, the sensor chip is used to detect parameters such as resistance, conductance, admittance and/or impedance to detect the EDA. The method of measuring the EDA using these parameters is known to the art and thus details thereof are not described herein.

As mentioned above, the conventional watch-type EDA sensor has the problems of unable for long-term and continuous measurement and poor signal quality corresponding to different electrode arrangements. Accordingly, the present disclosure further provides a ring-type EDA measurement device (e.g., referring to FIGS. 2A, 3A and 4A) that is worn on a user's finger to detect a voltage difference on different fingers to achieve the purpose of measuring the EDA. Because he EDA measurement device of the present disclosure is to detect signals on fingers, a better signal quality can be obtained compared with detecting signals on the user's wrist skin. Meanwhile, corresponding to different arranged positions of two electrodes, the present disclosure is adapted to the usage scenarios of active measurement and passive measurement. Finally, by electing a proper size, the ring-type structure can have high contact stability with the finger skin to improve the accuracy of measuring physiological data.

Although the disclosure has been explained in relation to its preferred embodiment, it is not used to limit the disclosure. It is to be understood that many other possible modifications and variations can be made by those skilled in the art without departing from the spirit and scope of the disclosure as hereinafter claimed.

1. An electrodermal activity (EDA) measurement device, configured to be worn on a finger of a user, and the EDA measurement device comprising:
a ring body, comprising an outer surface and an inner surface;
a first electrode, arranged at a first zone on the outer surface, and configured to contact a first finger of the user;
a second electrode, arranged at a second zone on the outer surface, and configured to contact a second finger of the user; and
an isolation zone, arranged on the outer surface, and configured to electrically isolate the first electrode and the second electrode.

2. The EDA measurement device as claimed in claim 1, wherein a surface of the first electrode and the second electrode is respectively a curved surface.

3. The EDA measurement device as claimed in claim 1, wherein a surface of the first electrode and the second electrode is respectively a flat surface.

4. The EDA measurement device as claimed in claim 1, wherein the first zone and the second zone are arranged as
upon the ring body being worn on a predetermined finger of the user at a predetermined angle, the first electrode and the second electrode respectively in contact with two adjacent fingers at two sides of the predetermined finger.

5. The EDA measurement device as claimed in claim 1, wherein the first zone and the second zone are arranged as
upon the ring body being worn on a predetermined finger of the user at a predetermined angle, one of the first and second electrodes in contact with one of two adjacent fingers at two sides of the predetermined finger, and the other one of the first and second electrodes not in contact with the other one of the two adjacent fingers at two sides of the predetermined finger.

6. The EDA measurement device as claimed in claim 1, further comprising a sensor chip configured to
provide a first voltage to the first electrode and provide a second voltage, different from the first voltage, to the second electrode in a first time interval, and
provide the second voltage to the first electrode and provide the first voltage to the second electrode in a second time interval.

7. The EDA measurement device as claimed in claim 6, wherein the sensor chip is further configured to transmit measurement data to a cellphone or a watch in a wireless manner.

8. An electrodermal activity (EDA) measurement device, configured to be worn on a finger of a user, and the EDA measurement device comprising:
a ring body, comprising an outer surface and an inner surface;
a first electrode, arranged at a first zone on the outer surface, and configured to contact a first finger of the user; and
a second electrode, arranged at a second zone on the inner surface, and configured to contact a second finger of the user.

9. The EDA measurement device as claimed in claim 8, wherein the second zone covers a part of or the whole of the inner surface.

10. The EDA measurement device as claimed in claim 8, wherein the first zone is arranged as
upon the ring body being worn on the second finger of the user at a predetermined angle, the first electrode in contact with one of two adjacent fingers at two sides of the second finger but not in contact with the other one of the two adjacent fingers at two sides of the second finger.

11. The EDA measurement device as claimed in claim 10, wherein a surface of the first electrode is a curved surface or a flat surface.

12. The EDA measurement device as claimed in claim 8, wherein the first zone is arranged as
upon the ring body being worn on the second finger of the user at a predetermined angle, the first electrode not in contact with any one of two adjacent fingers at two sides of the second finger.

13. The EDA measurement device as claimed in claim 8, wherein upon the ring body being worn on the second finger of the user at a predetermined angle, two opposite sides of the outer surface of the ring body corresponding to two adjacent fingers at two sides of the second finger are curved surfaces or flat surfaces.

14. The EDA measurement device as claimed in claim 8, further comprising a sensor chip configured to
provide a first voltage to the first electrode and provide a second voltage, different from the first voltage, to the second electrode in a first time interval, and
provide the second voltage to the first electrode and provide the first voltage to the second electrode in a second time interval.

15. An electrodermal activity (EDA) measurement device, comprising:
a first ring body, configured to be worn on a first finger of a user, and comprising a first electrode arranged on one of an inner surface and an outer surface of the first ring body and configured to contact the first finger or an adjacent finger of the first finger;
a second ring body, configured to be worn on a second finger of the user, and comprising a second electrode arranged on one of an inner surface and an outer surface of the second ring body and configured to contact the second finger or an adjacent finger of the second finger; and
a sensor chip, arranged on the first ring body or the second ring body, and electrically coupled to the first electrode and the second electrode.

16. The EDA measurement device as claimed in claim 15, wherein the sensor chip is configured to
provide a first voltage to the first electrode and provide a second voltage, different from the first voltage, to the second electrode in a first time interval, and
provide the second voltage to the first electrode and provide the first voltage to the second electrode in a second time interval.

17. The EDA measurement device as claimed in claim 15, wherein
the first finger and the second finger are two adjacent fingers of the user,
upon the first electrode being arranged at the outer surface of the first ring body, the first electrode is arranged at an opposite side of the second ring body, and
upon the second electrode being arranged at the outer surface of the second ring body, the second electrode is arranged at an opposite side of the first ring body.

18. The EDA measurement device as claimed in claim 17, wherein
a surface of the first electrode is a curved surface or a flat surface, and
a surface of the second electrode is a curved surface or a flat surface.

19. The EDA measurement device as claimed in claim 15, wherein
upon the first electrode being arranged at the inner surface of the first ring body, the first electrode covers a part of or the whole of the inner surface of the first ring body, and
upon the second electrode being arranged at the inner surface of the second ring body, the second electrode covers a part of or the whole of the inner surface of the second ring body.

20. The EDA measurement device as claimed in claim 15, wherein the sensor chip is further configured to transmit measurement data to a cellphone or a watch in a wireless manner.
